(19) United States
(12) Patent Application Publication
(10) Pub. No.: US 2009/0215888 A1
(43) Pub. Date: Aug. 27, 2009

(54) TOPICAL NAIL FORMULATION

(76) Inventors: Singh Jagat, Scarborough (CA); Dan McKay, Toronto (CA)

Correspondence Address: KATTEN MUCHIN ROSENMAN LLP;(C/O PATENT ADMINISTRATOR), 2900 K STREET NW, SUITE 200, WASHINGTON, DC, 20007-5118, US

(21) Appl. No.: 12/281,258

(22) PCT Filed: Mar. 1, 2007
(86) PCT No.: PCT/CA07/00323

§ 371 Date: Nov. 25, 2008

Related U.S. Application Data
(60) Provisional application No. 60/778,103, filed on Mar. 2, 2006.

(51) Int. Cl. A61K 31/27

(52) U.S. Cl. 514/479

(57) ABSTRACT

The present invention relates to a topical nail formulation comprising at least one active agent (e.g., tolnaftate) and at least one nail penetration enhancer selected from the group consisting of a fatty acid, an azone derivative, and mixtures thereof. The invention also relates to the use of said topical nail formulation for the treatment of nail disorders such as onychomycosis. The invention further provides a method of enhancing the nail flux of an active agent by using a topical nail formulation containing the said penetration enhancer.

### FIELD OF THE INVENTION

The present invention provides a topical nail formulation and more particularly a topical nail formulation including at least one penetration enhancer selected from the group consisting of a fatty acid, an azone-related compound and mixtures thereof.

### BACKGROUND OF THE INVENTION

Human nails can suffer from a number of disorders including discolouration due to smoking or use of systemic drugs, brittleness through repeated exposure to certain chemical as well as infections.

The two most common diseases affecting the nail are onychomycosis (fungal infections of the nail plate and/or nail bed) and psoriasis. Nail fungal infections are usually treated with oral antifungal medications which can be associated with undesirable side effects due to systemic distribution of a drug. Additionally, treating nail infections with oral drugs typically takes many months of therapy which can lead to poor compliance. Systemically delivered drugs are also of limited benefit in patients with an impaired host immune response. Additionally lateral disease of the nail can lead to treatment failure with systemic medications.

Topical therapies offer a number of advantages including ease of administration, avoidance of systemic distribution and first pass metabolism and targeting of the drug to the local site of action. However, drug diffusion into the keratinized nail plate is poor. Although some topical therapies for nail fungal infections exist they have limited efficacy and there is considerable room for improvement.

The human nail provides an even more formidable barrier to entry of foreign substances than does the skin. Although the nail plate derives from epidermal tissue as does the stratum corneum, there are considerable physical and chemical differences between the two. The nail plate is approximately 25 layers of keratinized cells fused into three layers: a dense and hard dorsal plate, a thick intermediate plate and a thin ventral plate. The thickness of a human nail is between 0.5-1.0 mm, which is as much as 100 times thicker than the stratum corneum (typically 10-40 um). Additionally the lipid content of the nail is between 0.1 and 1% whereas the lipid content of the stratum corneum is 10-20%.

The intercellular lipid domains of the stratum corneum are a key transport pathway for skin penetration. The mechanism for a vast majority of skin penetration enhancers involves a disruption of the lipid domains or pathways in the stratum corneum. These penetration enhancers are unlikely to have the same penetration enhancement effect on the nail since the nail contains much less lipid and less developed lipid pathways. [Sun et al.: Nail Penetration in Percutaneous Absorption, 3^{rd} Edition, Bronough R., Maibach H. (Eds.), Mercel Dekker Inc. 759-779].

### SUMMARY OF THE INVENTION

In one aspect the present invention provides a topical nail formulation comprising at least one active agent and at least one penetration enhancer selected from the group consisting of a fatty acid, an azone-related compound and mixtures thereof.

In an alternative embodiment the present invention provides a topical nail formulation comprising at least one antifungal agent and a penetration enhancer selected from oleic acid, azone and mixtures thereof.

In a further embodiment the present invention provides a topical nail formulation comprising at least one of a fatty acid, an azone-related compound and mixtures thereof.

In a further aspect the present invention provides a method of treating or ameliorating a nail condition comprising the steps of administering to a nail in need of such treatment a therapeutically effective amount of the nail formulation described herein.

In a further aspect the present invention provides a method for enhancing the nail flux of an active agent comprising the steps of administering to a nail the topical formulation described herein with a therapeutically effective amount of an active agent.

In an alternative aspect the present invention provides a method of delivering an active agent into or through the nail comprising the steps of administering to a nail a therapeutically effective amount of the nail formulation described herein.

### BRIEF DESCRIPTION OF THE DRAWINGS

The present invention will be discussed in further detail below with reference to the accompanying drawings in which:

FIG. 1 graphically illustrates the cumulative penetration results of the formulations described in example 1; and

FIG. 2 graphically illustrates the total percutaneous absorption of the antifungal of the formulations described in example 1.

### DETAILED DESCRIPTION OF THE PREFERRED EMBODIMENTS

The present invention will now be described in further detail with reference to the accompanying Figures where appropriate.

In one embodiment of the present invention a topical nail formulation is provided comprising at least one active agent and at least one penetration enhancer selected from the group consisting of a fatty acid, an azone-related compound and mixtures thereof.

In an alternative embodiment the present invention provides a topical nail formulation comprising at least one of a fatty acid, an azone-related compound and mixtures thereof.

The term ‘penetration enhancer’ is used herein to refer to an agent that improves the transport of an active agent, or medicine, into or through the nail. A ‘penetration enhancer’ is used to assist in the delivery of an active agent directly or indirectly to the site of the disease.

The term ‘composition’ used herein may be used interchangeably with the term ‘formulation’.

The terms “azone” and “1-dodecyl azacycloheptan-2-one” may be used interchangeably herein.

The term “onychomycosis” refers to a fungal infection of either the nail plate and/or the nail bed.

The formulations described herein are used to treat conditions of or related to the nail. Such conditions include but are not limited to infections, inflammation, psoriasis, paronychia, benign and malignant nail tumors and aesthetic conditions. In particular, the formulations described herein are used to treat onychomycosis.

Examples of active agents that may be included in the formulations described herein include, but are not limited to, antibiotics, antifungals, anti-inflammatories, antipsoriatic, anticancers, and other active agents such as steroids, methotrexate, cyclosporin, retinoids, pharmaceutically acceptable salts thereof and mixtures thereof.

Examples of antibiotics include, but are not limited to nystatin, natamycin, hitachimycin, pecilocin, mepartricin, pyrrolnitrin and griseofulvin. Antifungal agents include, but are not limited to, azoles, allylamines, morpholines, polyenes, tetraenes, pyrimidines, thiocarbamates, sulfonamides, organic acids, hydroxides, echinocandins and other agents.

Examples of azole compounds include imidazoles and triazole derivatives. including but not limited to ketoconazole, miconazole, bifonazole, butoconazole, clotrimazole, clomidazole, croconazole, eberconazole, econazole, fenticonazole, fluconazole, flutrimazole, isoconazole, itraconazole, ketoconazole, lanoconazole, neticonazole, omoconazole, oxiconazole, setraconazole, sulconazole, terconazole, tiabendazole, tioconazole.

Examples of allylamine compounds include, but are not limited to, terbinafine and natrifine. Examples for morphonlines include amorolfine. Examples for polyenes include, but are not limited to amphotericin B, nystatin, and natamaycin. Examples for pyrimidine include, but are not limited to flucytosine and 5-fluorocytosine. Examples of tetraene include, but are not limited to natamycin. Thiocarbamate includes, but is not limited to tolnaftate. Examples of sulphonamide include, but are not limited to mafenide and dapsone. Examples of organic acid include, but is not limited to undecylenic acid. Examples of hydroxides include, but is not limited to potassium hydroxide. Examples of echinocandins include, but is not limited to anidulafungin. Other suitable agents include bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, potassium iodide, butenafine, ciclopirox, ciloquinol (iodochlorhydroxyquin), haloprogin, aluminum chloride, potassium permanganate, selenium sulphide, salicylic acid, sulphacetamide, benzoic acid, silver sulfadiazine and zinc pyruthione.

Additional examples of agents that may be used in the nail formulation described herein include anti-neoplastic agents, such as adriamycine, cyclophosphamide, methotrexate; anticancer agents, such as paclitaxel, N-[[(substituted phenyl)amino]carbonyl]alkylsulfonamides, 5-fluorouracil; antipsoriatics, such as coal tar, flurandrenolide, and dithranol; immune response steroidal anti-inflammatory agents, such as hydrocortisone, dioxyanthranol, and betamethasone; non-steroidal anti-inflammatory agents (NSAIDs), such as celecoxib, diclofenac, diflunisal, etodolac, fenoprofen; flurbiprofen, ibuprofen, ketoprofen, ketorolac, indomethacin, lumiracoxib meclofenamate; mefenamic acid, meloxicam, nabumetone, naproxen, oxaprozin, oxyphenbutazone; phenylbutazone, piroxicam, rofecoxib, salsalate, sulindac, tolmetin, valdecoxib, salicylates, zomepirac or local anesthetics, such as articaine, benzocaine, bupivacaine, capsaicin, cinchocaine, chloroprocaine, dyclonine, etidocaine, ethyl chloride, levobupivacaine, lidocaine, mepivacaine, phenol, procaine, prilocaine, ropivacaine, tetracaine, analgesics and analgesic combinations, such as acetaminophen, aspirin.

It will be understood that one or more of the actives described above may exist, and be used, in different polymorphic or isomeric forms. In addition, one or more of the actives may be used in different salt forms.

As described above, in one embodiment the present invention provides a topical nail formulation comprising at least one active agent and at least one penetration enhancer selected from the group consisting of a fatty acid, an azone-related compound and mixtures thereof.

Preferably the active agent is an antifungal agent. Preferably the antifungal agent is selected from the group consisting of ketoconazole, miconazole, bifonazole, butoconazole, clomidazole clotrimazole, croconazole, eberconazole, econazole, fenticonazole, flutimazole, isoconazole, ketoconazole, lanoconazole, neticonazole, omoconazole, oxiconazole, setraconazole, sulconazole, tioconazole, fluconazole, itraconazole, terconazole, terbinafine, natrifine, amorolfine, amphotericin B, nystatin, natamaycin, flucytosine, griseofulvin, potassium iodide, butenafine, ciclopirox, ciloquinol (iodochlorhydroxyquin), haloprogin, tolnaftate, aluminum chloride, undecylenic acid, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, sulphacetamide, benzoic acid and pharmaceutically acceptable salts thereof. More preferably the antifungal agents is selected from the group consisting of econazole, terbinafine, butenafine, tolnaftate and pharmaceutically acceptable salts thereof. More preferably the antifungal agent is tolnaftate or a pharmaceutically acceptable salt thereof.

In one embodiment of the topical formulation described above the penetration enhancer is a fatty acid selected from the group consisting of alkanoic acids, capric acid, diacid, ethyloctadecanoic acid, hexanoic acid, lactic acid, lauric acid, linoelaidic acid, linoleic aid, linolenic acid, neodecanoic acid, oleic acid, palmitic acid, pelargonic acid, propionic acid, vaccenic acid and mixtures thereof. Preferably the penetration enhancer is oleic acid.

In an alternative embodiment of the topical formulation described above the penetration enhancer is an azone-related compound selected from the group consisting of N-acyl-hexahydro-2-oxo-1H-azepines, N-alkyl-dihydro-1,4-oxazepine-5,7-diones, N-alkymorpholine-2,3-diones, N-alkylmorpholine-3,5-diones, azacycloalkane derivatives (-ketone, -thione), azacycloalkenone derivatives, 1-[2-(decylthio)ethyl]azacyclopentan-2-one, N-(2,2-dihydroxyethyl)dodecylamine, 1-dodecanoylhexahydro-1-H-azepine, 1-dodecyl azacycloheptan-2-one, N-dodecyl diethanolamine, N-dodecyl-hexahydro-2-thio-1H-azepine, N-dodecyl-N-(2-methoxyethyl)acetamide, N-dodecyl-N-(2-methoxyethyl)isobutyramide, N-dodecyl-piperidine-2-thione, N-dodecyl-2-piperidinone, N-dodecyl pyrrolidine-3,5-dione, N-dodecyl pyrrolidine-2-thione, N-dodecyl-2-pyrrolidone, 1-farnesylazacycloheptan-2-one, 1-farnesylazacyclopentan-2-one, 1-geranylazacycloheptan-2-one, 1-geranylazacyclopentan-2-one, hexahydro-2-oxo-azepine-1-acetic acid esters, N-(2-hydroxyethyl)-2-pyrrolidone, 1-laurylazacycloheptane, 2-(1-nonyl)-1,3-dioxolane, 1-N-octylazacyclopentan-2-one, N-(1-oxododecyl)-hexahydro-1H-azepine, N-(1-oxododecyl)-morpholines, 1-oxohydrocarbyl-substituted azacyclohexanes, N-(1-oxotetradecyl)-hexahydro-2-oxo-1H-azepine, N-(1-thiododecyl)-morpholines and mixtures thereof. Preferably the penetration enhancer is 1-dodecyl azacycloheptan-2-one, i.e. azone.

In an alternative embodiment of the present invention described above the topical formulation includes oleic acid in a concentration from about 1% to about 50% by weight of the formulation. Preferably the concentration of oleic acid is from about 1% to about 30% by weight of the formulation. More preferably the concentration of oleic acid is from about 1% to about 10% by weight of the formulation. In a preferred embodiment the concentration of oleic acid is from about 1% to about 5% by weight of the formulation.

In an alternative embodiment of the present invention described above the topical formulation includes azone in a concentration from about 1% to about 50% by weight of the formulation. Preferably the concentration of azone is from about 1% to about 30% by weight of the formulation. More preferably the concentration of azone is from about 1% to about 10% by weight of the formulation. In a preferred embodiment the concentration of azone is from about 1% to about 5% by weight of the formulation.

In a further embodiment of the present invention the topical formulation described above further comprises at least one organic sulfoxide. The at least one organic sulfoxide may be selected from the group consisting of a dialkyl sulfoxide compound, a cyclic sulfoxide compound and mixtures thereof. Preferably the organic sulfoxide is selected from the group consisting of dimethyl sulfoxide, 1-methylpropyl methyl sulfoxide, 1,1-dimethylpropyl methyl sulfoxide, 1,1-dimethylethyl methyl sulfoxide, 1-methylbutyl methyl sulfoxide, 1,1-dimethylbutyl methyl sulfoxide, 1-ethylbutyl methyl sulfoxide, 1-propylpentyl methyl sulfoxide, trimethylene sulfoxide, 1-propyltrimethylene sulfoxide, 1-butyltrimethylene sulfoxide, thiophene oxide, methyl ethyl sulfoxide, methyl ethylene sulfoxide, 2-hydroxyundecyl methyl sulfoxide, N-decylmethyl sulfoxide and mixtures thereof. More preferably the organic sulfoxide is selected from the group consisting of dimethyl sulfoxide, 2-hydroxyundecyl methyl sulfoxide, decylmethyl sulfoxide and mixtures thereof. In a preferred embodiment the organic sulfoxide is dimethyl sulfoxide.

In the embodiment that includes at least one organic sulfoxide, the organic sulfoxide may be present between about 1% and about 50% by weight of the formulation. In a preferred embodiment the at least one organic sulfoxide is present between about 1% and about 25% by weight of the formulation. In one embodiment the topical nail formulation includes about 5% of the at least one organic sulfoxide by weight of the formulation. In an alternative embodiment the topical nail formulation includes about 1% w/w of the at least one organic sulfoxide.

The topical nail formulation of the present invention may be administered in a variety of different delivery forms, for example but not limited to, solutions, gels, lacquers, lotions or cream formulations. A person skilled in the art will know the type of pharmaceutically acceptable carriers and excipients that may be used to prepare each of these types of formulations.

In order to provide a gel formulation a hydrophilic gelling agent may be used such as vinyl acetate copolymers, cellulose derivatives, polyvinyl pyrrolidone and carboxyvinyl copolymers. Such hydrophilic gelling agents may be present in the formulation in the range of between about 1% to about 50% w/w.

In order to provide a lacquer a water insoluble film former may be used such as acrylate polymers, methacrylate polymers, copolymers of alkyvinyl ether and maleic anhydrides. Such film formers may be present in the formulation in the range between about 1% and about 50% w/w. In addition, plasticizers such as phthalic acid ester, glycol, triacetin, castor oil and mixtures thereof may be used. Such plasticizers may be present in the formulation in the range of between about 1% to about 50% w/w.

In order to provide a lotion or cream product the following excipients may be used: fatty alcohols such as cetyl alcohol, stearyl alcohol; fatty acids such as stearic acid; fatty acid esters such as glyceryl monostearate and sorbitol monooleate; surfactants such as Tween and sodium lauryl sulfate. These excipients may be present in the formulation in the range of between about 1% to about 30% w/w.

It will be understood that the above examples are not meant to be limiting but merely provide examples of known excipients that may be used in the present invention.

Examples of suitable excipients that may be used to provide a solution include, but are not limited to, solvents such as polyethylene glycols, propylene glycol, isopropyl alcohol, ethanol, ethyl acetate, n-butyl acetate, water, and phosphate buffered saline (PBS). Other examples of suitable excipients for creating gels, solutions, creams, lacquers are listed in Remington's Pharmaceutical Sciences, 18th Edition, Ed. Alfonso Gennaro, Mack Publishing Co. Easton, Pa., 1995 and Handbook of Pharmaceutical Excipients, 3rd Edition, Ed. Arthur H. Kibbe, American Pharmaceutical Association, Washington D.C. 2000, both of which are incorporated herein by reference.

In one embodiment of the present invention the topical nail formulation includes at least one active agent, a penetration enhancer selected from azone and oleic acid, ethanol, propylene glycol, and polyethylene glycol 300. The topical nail formulation may also optionally include cosmetic ingredients such as moisturizers, humectants or emollients. Suitable cosmetic ingredients are listed in the International Cosmetic Ingredient Dictionary, 11^{th} Edition published by the Cosmetic, Toiletry, and Fragrance Association, Inc. 1101 17th Street, NW, Suite 300, Washington D.C. 20036-4702.

In a further embodiment of the present invention a topical nail formulation is provided comprising:
(i) between about 1% to about 10% w/w azone;
(ii) between about 1% to about 25% w/w propylene glycol;
(iii) between about 1% to about 15% w/w glycerine;
(iv) between about 1% to about 25% w/w ethanol;
(v) between about 1% to about 80% w/w polyethylene glycol 300; and
(vi) at least one active agent.

In a preferred embodiment of the formulation the at least one active agent is an antifungal agent or a pharmaceutically acceptable salt thereof, preferably tolnaftate, and the azone is present between about 1% to about 5% by weight of the formulation.

In an alternative embodiment a topical nail formulation is provided comprising:
(i) between about 1% to about 10% w/w oleic acid;
(ii) between about 1% to about 25% w/w propylene glycol;
(iii) between about 1% to about 15% w/w glycerine;
(iv) between about 1% to about 25% w/w ethanol;
(v) between about 1% to about 80% w/w polyethylene glycol 300; and
(vi) at least one active agent.

In a preferred embodiment of the formulation the at least one active agent is an antifungal agent or a pharmaceutically acceptable salt thereof, preferably tolnaftate, and the oleic acid is present between about 1% to about 5% by weight of the formulation.

In a further embodiment the topical nail formulation comprises:
i) about 2% w/w tolnaftate;
ii) about 12% w/w propylene glycol; iii) about 12% w/w glycerine;
iv) about 20% w/w ethanol;
v) about 5% w/w oleic acid; and
vi) polyethylene glycol 300 qs.

In a further embodiment the above formulation includes about 10% w/w oleic acid. In an alternative embodiment the above formulation includes about 5% w/w azone in place of the oleic acid. In a further embodiment the above formulation includes about 10% w/w azone in place of the oleic acid. The formulation may also include a mixture of oleic acid and azone.

In another aspect of the present invention a method of treating or ameliorating a nail condition is provided comprising the steps of administering to a nail in need of such treatment a therapeutically effective amount of the nail formulation described herein. In a preferred embodiment the nail condition is onychomycosis.

In a further aspect of the present invention a method for enhancing the nail flux of an active agent, or pharmaceutically acceptable salt thereof, is provided comprising the steps of administering to a nail the topical formulation described herein with a therapeutically effective amount of the active agent. In a further embodiment the active agent is an antifungal agent.

In an alternative aspect the present invention provides a method of delivering an active agent, or pharmaceutically acceptable salt thereof, into or through the nail comprising the steps of administering to a nail a therapeutically effective amount of the nail formulation described herein.

### Example 1

### Topical Nail Formulation Comprising Tolnaftate

Normal human cadaver fingernails were obtained from donors and consisted of the first, second, third, and fourth fingernails from each. The nails were without obvious signs of disease, and were obtained within seven days of death. At collection, the nails were sealed in a water-impermeable plastic bag, and stored at <−20° C. until the day of the experiment. Prior to use they were thawed at room temperature.

Prior to use, the nails were cleared of any underlying tissue and rinsed in tap water to remove any adherent blood or other material from their surfaces. When necessary, nails were trimmed to fit the chambers. Final nail thickness, dimensions and weight were measured using a ruler, micrometer and balance, and recorded.

Nail sections were mounted onto modified Franz diffusion cells specially designed to support human nails, and sealed into place with silicone sealant. Chambers were selected with either a 7 mm or 9 mm mounting surface based on the size of the nail being mounted. The receptor chamber was filled to capacity (˜4 mLs; each chamber volume was recorded) with a solution of 0.1× Phosphate Buffered Saline (PBS) with 0.1% Volpo. The nail surface was open to ambient laboratory environment. The cells were then placed in a diffusion apparatus in which the dermal receptor solution temperature was maintained at 32±1.0° C. The diffusion apparatus consisted of an open humidity environment such that the humidity was within the range of approximately 30-70%.

Dosing and Sample: Prior to administration of the topical test formulations, provided in Table 1, to the nail sections, the receptor solution was replaced with a fresh solution of 0.1× PBS with 0.1% Volpo. Subsequently, each test product was applied to duplicate nail sections within the same donor at a dose of 6.4 μL/9 mm nail or 3.8 μL/7 mm nail (both targeted to be 10 μL/cm²) using a calibrated positive displacement pipette. The same applied dose was repeated twice each day (8 to 10 hours apart) for 14 consecutive days. Prior to each dose application the nail was gently washed with soap (1% Sodium lauryl sulfate; SLS) in water, and water rinsed, using cotton-tipped swabs.

Nail Samples: At 24 hr intervals, for 15 days, the receptor solution was removed in its entirety, replaced with fresh receptor solution, and a predetermined volume aliquot saved for subsequent analysis. Following the last collected sample, the nail was removed from the chamber, gently rinsed with 1% SLS and water, and then processed for transverse sectioning by microtome.

Nail sectioning was conducted by punching out the center ˜0.5 cm diameter of the dosed region of the nail and slicing the punch horizontal to the nail surface using a manual microtome. Approximately 9-15 sections were collected from each nail, and divided into the first ⅓ number of sections, the second ⅓ number of sections and the final ⅓ number of sections. The sections were collected, weighed by group, and extracted in acetonitrile/water (1:1) for subsequent analysis. Although targeted for ⅓ portions of the nail, based on weight of each portion, on average, the top portions contained 20% of the total nail weight, the middle portion contained 30% of the total nail weight, and the bottom portion contained 50% of the total nail weight.

Analytical Methods: Quantification of Tolnaftate was by High Performance Liquid Chromatography-Mass Spectroscopy (HPLC-MS). Briefly, HPLC was conducted on an Agilent 1100 Series HPLC system with a diode array detector, and an Agilent 1100 Series MSD. A solvent system consisting of 20% 20 mM Ammonium acetate in water, pH 2.8 with TFA, and 80% 90:10 Acetonitrile:water was run through a C18 Luna column (50×3 mm, 3μ, Gemini Phenomenex Inc.) at a flow rate of 1.0 mL/min (1.1 minute run duration). Ten microliters of sample were injected. Peak areas were quantified to concentration using an external standard curve prepared from the neat standard.

TABLE 1
Topical nail formulations that were tested.

| | | | Propylene | | EtOH | Oleic | | PEG |
|---|---|---|---|---|---|---|---|---|
| Formulation | DMSO | Tolnaftate | Glycol | Glycerine | 95% | Acid | Azone | 300 |
| OC1 | 0.0 | 2.0 | 12.0 | 12.0 | 20.0 | 5.0 | — | qs to 25 |
| Concentration | | | | | | | | mL |
| (% w/v) | | | | | | | | |
| OC2 | 0.0 | 2.0 | 12.0 | 12.0 | 20.0 | 10.0 | — | qs to 25 |
| Concentration | | | | | | | | mL |
| (% w/v) | | | | | | | | |
| DM1 | 5.0 | 2.0 | 11.2 | 11.2 | 11.79 | 0.0 | — | qs to 25 |
| Concentration | | | | | | | | mL |
| (% w/v) | | | | | | | | |
| DO1 | 45.5 | 2.0 | 11.2 | 11.2 | 11.79 | 5.0 | — | qs to 25 |
| Concentration | | | | | | | | mL |
| (% w/v) | | | | | | | | |
| DO2 | 45.5 | 2.0 | 11.2 | 11.2 | 11.79 | 10.0 | — | qs to 25 |
| Concentration | | | | | | | | mL |
| (% w/v) | | | | | | | | |
| DO3 | 5.0 | 2.0 | 11.2 | 11.2 | 11.79 | 5.0 | — | qs to 25 |
| Concentration | | | | | | | | mL |
| (% w/v) | | | | | | | | |
| DO4 | 5.0 | 2.0 | 11.2 | 11.2 | 11.79 | 10.0 | — | qs to 25 |
| Concentration | | | | | | | | mL |
| (% w/v) | | | | | | | | |
| AZ1 | 0.0 | 2.0 | 11.2 | 11.2 | 11.79 | — | 1.0 | qs to 25 |
| Concentration | | | | | | | | mL |
| (% w/v) | | | | | | | | |
| AZ5 | 0.0 | 2.0 | 11.2 | 11.2 | 11.79 | — | 5.0 | qs to 25 |
| Concentration | | | | | | | | mL |
| (% w/v) | | | | | | | | |

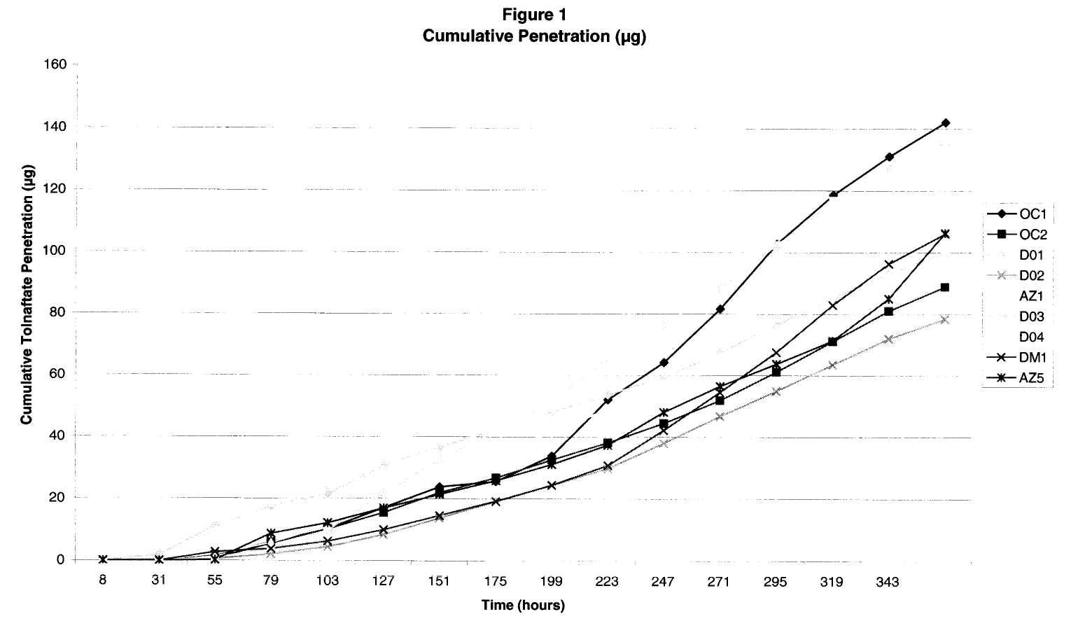

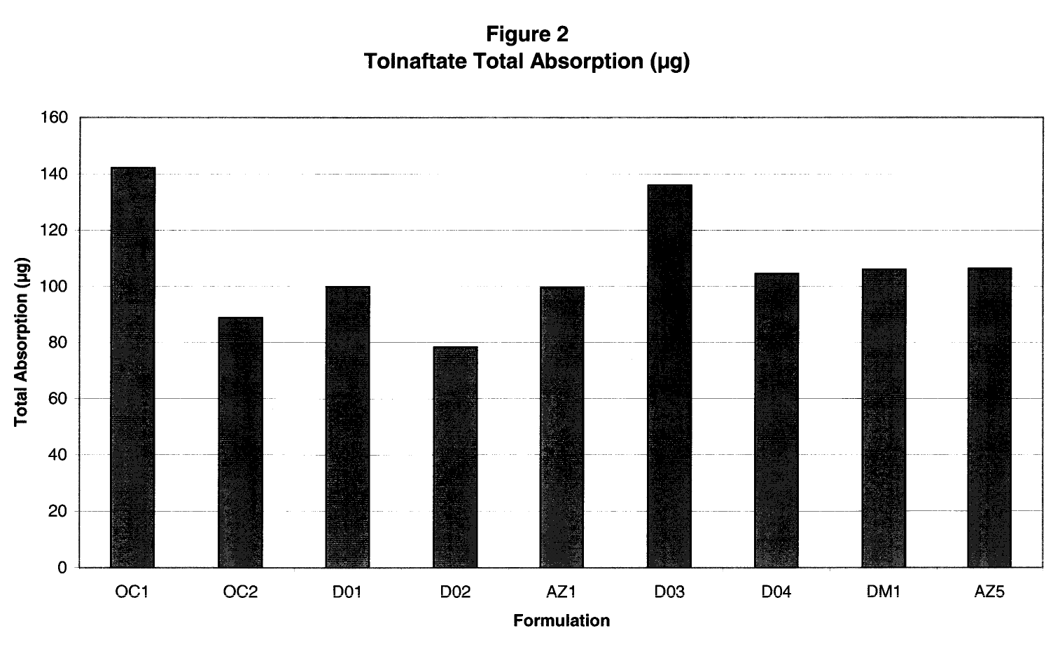

Results: The results for each formulation tested, as described above, are provided in Tables 2 and 3 and graphically illustrated in FIGS. 1 and 2. FIG. 1 illustrates the cumulative penetration results over time for each formulation tested. FIG. 2 illustrates the total nail absorption of tolnaftate for each formulation tested.

TABLE 2
Cumulative Penetration (μg) of Tolnaftate through Human Cadaver Nails over
15 Days from Twice/Daily Application (Mean ± SD, n = 3 Donors)

| Time | | | | | | | | | |
|---|---|---|---|---|---|---|---|---|---|
| (hr)* | OC1 | OC2 | D01 | D02 | AZ1 | D03 | D04 | DM1 | AZ5 |
| 8 | 0 | 0.041 | 1.871 | 0.222 | 0 | 0 | 0.04 | 0 | 0 |
| 31 | 0.752 | 1.764 | 11.092 | 0.579 | 9.669 | 0.861 | 2.066 | 2.778 | 0.249 |
| 55 | 5.506 | 5.922 | 17.568 | 2.076 | 18.318 | 3.931 | 5.696 | 3.84 | 8.708 |
| 79 | 10.265 | 10.18 | 21.378 | 4.431 | 23.613 | 9.799 | 11.463 | 6.266 | 12.2 |
| 103 | 17.076 | 15.508 | 30.857 | 8.377 | 29.003 | 21.556 | 18.905 | 9.998 | 16.942 |
| 127 | 23.841 | 21.969 | 36.825 | 13.775 | 33.985 | 32.827 | 26.669 | 14.582 | 21.39 |
| 151 | 25.562 | 26.786 | 42.199 | 19.076 | 36.217 | 41.93 | 34.17 | 19.202 | 25.883 |
| 175 | 33.891 | 32.588 | 48.112 | 24.337 | 41.263 | 51.763 | 41.568 | 24.404 | 31.084 |
| 199 | 52.086 | 38.182 | 53.657 | 29.788 | 49.259 | 65.583 | 48.915 | 30.775 | 37.355 |
| 223 | 64.241 | 44.493 | 59.615 | 37.985 | 56.391 | 76.547 | 56.787 | 42.203 | 48.065 |
| 247 | 81.678 | 51.891 | 67.374 | 46.803 | 62.65 | 88.484 | 66.036 | 54.55 | 56.516 |
| 271 | 102.751 | 61.152 | 76.521 | 54.952 | 73.873 | 102.354 | 75.533 | 67.593 | 63.744 |
| 295 | 118.551 | 71.093 | 85.087 | 63.5 | 84.337 | 116.764 | 86.951 | 82.915 | 71.31 |
| 319 | 131.061 | 81.014 | 93.167 | 71.986 | 92.712 | 127.485 | 96.386 | 96.336 | 84.996 |
| 343 | 142.119 | 88.834 | 99.907 | 78.364 | 99.549 | 135.996 | 104.645 | 106.056 | 106.37 |

TABLE 3
Summary of Tolnaftate Total Absorption - Percutaneous Absorption
of Tolnaftate through Human Cadaver Fingernails over 15
Days from Twice/Day Applications (Mean ± SD, n = 3
Donors/formulation) as Total Mass (μg)

| Formulation | Total Absorption (μg) |
|---|---|
| OC1 | 142.12 ± 52.81 |
| OC2 | 88.83 ± 35.80 |
| D01 | 99.91 ± 84.19 |
| D02 | 78.36 ± 28.34 |
| AZ1 | 99.55 ± 58.62 |
| D03 | 136.00 ± 63.28 |
| D04 | 104.65 ± 44.87 |
| DM1 | 106.06 ± 59.70 |
| AZ5 | 106.37 ± 70.55 |

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments. Further, all of the claims are hereby incorporated by reference into the description of the preferred embodiments.

Any and all publications, patents and patent applications referred to herein are incorporated by reference in their entirety to the same extent as if each individual publication, patent or patent application was specifically and individually indicated to be incorporated by reference in its entirety.

What is claimed is:

1. A topical nail formulation comprising at least one active agent and at least one penetration enhancer selected from the group consisting of a fatty acid, an azone-related compound and mixtures thereof.

2. The topical nail formulation defined in claim 1, wherein the at least one active agent is selected from the group consisting of an antifungal agent, an anti-inflammatory agent, an anti-cancer agent, an antipsoriatic agent and pharmaceutically acceptable salts thereof.

3. The topical nail formulation defined in claim 2, wherein the antifungal agent is selected from the group consisting of ketoconazole, miconazole, bifonazole, butoconazole, clotrimazole, croconazole, eberconazole, econazole, fenticonazole, flutimazole, isoconazole, ketoconazole, lanoconazole, neticonazole, omoconazole, oxiconazole, setraconazole, sulconazole, tioconazole, fluconazole, itraconazole, terconazole, terbinafine, natrifine, amorolfine, amphotericin B, nystatin, natamaycin, flucytosine, griseofulvin, potassium iodide, butenafine, ciclopirox, ciloquinol (iodochlorhydroxyquin), haloprogin, tolnaftate, aluminum chloride, undecylenic acid, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, sulphacetamide, benzoic acid and pharmaceutically acceptable salts thereof.

4. The topical nail formulation defined in claim 2, wherein the antifungal agents is selected from the group consisting of econazole, terbinafine, butenafine, tolnaftate and pharmaceutically acceptable salts thereof.

5. The topical nail formulation defined in claim 2, wherein the antifungal agent is tolnaftate or a pharmaceutically acceptable salt thereof.

6. The topical nail formulation defined in claim 1, wherein the fatty acid is selected from the group consisting of alkanoic acids, capric acid, diacid, ethyloctadecanoic acid, hexanoic acid, lactic acid, lauric acid, linoelaidic acid, linoleic aid, linolenic acid, neodecanoic acid, oleic acid, palmitic acid, pelargonic acid, propionic acid, vaccenic acid and mixtures thereof.

7. The topical nail formulation defined in claim 1, wherein the fatty acid is oleic acid.

8. The topical nail formulation defined in claim 1, wherein the azone-related compound is selected from the group consisting of N-acyl-hexahydro-2-oxo-1H-azepines, N-alkyl-dihydro-1,4-oxazepine-5,7-diones, N-alkymorpholine-2,3-diones, N-alkylmorpholine-3,5-diones, azacycloalkane derivatives (-ketone, -thione), azacycloalkenone derivatives, 1-[2-(decylthio)ethyl]azacyclopentan-2-one, N-(2,2-dihydroxyethyl)dodecylamine, 1-dodecanoylhexahydro-1-H-azepine, 1-dodecyl azacycloheptan-2-one, N-dodecyl diethanolamine, N-dodecyl-hexahydro-2-thio-1H-azepine, N-dodecyl-N-(2-methoxyethyl)acetamide, N-dodecyl-N-(2-methoxyethyl)isobutyramide, N-dodecyl-piperidine-2-thione, N-dodecyl-2-piperidinone, N-dodecyl pyrrolidine-3,5-dione, N-dodecyl pyrrolidine-2-thione, N-dodecyl-2-pyrrolidone, 1-farnesylazacycloheptan-2-one, 1-farnesylazacyclopentan-2-one, 1-geranylazacycloheptan-2-one, 1-geranylazacyclopentan-2-one, hexahydro-2-oxo-azepine-1-acetic acid esters, N-(2-hydroxyethyl)-2-pyrrolidone, 1-laurylazacycloheptane, 2-(1-nonyl)-1,3-dioxolane, 1-N-octylazacyclopentan-2-one, N-(1-oxododecyl)-hexahydro-1H-azepine, N-(1-oxododecyl)-morpholines, 1-oxohydrocarbyl-substituted azacyclohexanes, N-(1-oxotetradecyl)-hexahydro-2-oxo-1H-azepine, N-(1-thiododecyl)-morpholines and mixtures thereof.

9. The topical nail formulation defined in claim 1, wherein the azone-related compound is 1-dodecyl azacycloheptan-2-one.

10. The topical nail formulation defined in claim 1, wherein the concentration of the penetration enhancer is from about 1% to about 50% by weight of the composition.

11. The topical nail formulation defined in claim 1, wherein the concentration of the penetration enhancer is from about 1% to about 30% by weight of the concentration.

12. The topical nail formulation defined in claim 1, wherein the concentration of the penetration enhancer is from about 1% to about 10% by weight of the concentration.

13. The topical nail formulation defined in claim 1, wherein the concentration of the penetration enhancer is from about 1% to about 5% by weight of the concentration.

14. The topical nail formulation defined in claim 1, further comprising at least one organic sulfoxide.

15. The topical nail formulation defined in claim 14, wherein the at least one organic sulfoxides is selected from the group consisting of dimethyl sulfoxide, 1-methylpropyl methyl sulfoxide, 1,1-dimethylpropyl methyl sulfoxide, 1,1-dimethylethyl methyl sulfoxide, 1-methylbutyl methyl sulfoxide, 1,1-dimethylbutyl methyl sulfoxide, 1-ethylbutyl methyl sulfoxide, 1-propylpentyl methyl sulfoxide, trimethylene sulfoxide, 1-propyltrimethylene sulfoxide, 1-butyltrimethylene sulfoxide, thiophene oxide, methyl ethyl sulfoxide, methyl ethylene sulfoxide, 2-hydroxyundecyl methyl sulfoxide, N-decylmethyl sulfoxide and mixtures thereof

16. The topical nail formulation defined in claim 14, wherein the at least one organic sulfoxide is dimethyl sulfoxide.

17. A topical nail formulation comprising at least one antifungal agent and a penetration enhancer selected from oleic acid, azone and mixtures thereof.

18. The topical formulation defined in claim 17, wherein the antifungal agent is selected from the group consisting of azoles, allylamines, morpholines, polyenes, tetraenes, pyrimidines, thiocarbamates, sulfonamides, organic acids, hydroxides, echinocandins and pharmaceutically acceptable salts thereof.

19. The topical formulation defined in claim 17, wherein the antifungal agent is selected from the group consisting of econazole, terbinafine, butenafine, tolnaftate and pharmaceutically acceptable salts thereof.

20. The topical formulation defined in claim 17, wherein the antifungal agent is tolnaftate or a pharmaceutically acceptable salt thereof.

21. The topical formulation defined in claim 17, wherein the concentration of oleic acid is from about 1% to about 50%.

22. The topical formulation defined in claim 17, wherein the concentration of oleic acid is from about 1% to about 30%.

23. The topical formulation defined in claim 17, wherein the concentration of oleic acid is from about 1% to about 10%.

24. The topical formulation defined in claim 17, wherein the concentration of oleic acid is from about 1% to about 5%.

25. The topical formulation defined in claim 17, wherein the concentration of oleic acid is about 5%.

26. The topical formulation defined in claim 17, wherein the concentration of azone is from about 1% to about 50%.

27. The topical formulation defined in claim 17, wherein the concentration of azone is from about 1% to about 30%.

28. The topical formulation defined in claim 17, wherein the concentration of azone is from about 1% to about 10%.

29. The topical formulation defined in claim 17, wherein the concentration of azone is from about 1% to about 5%.

30. The topical formulation defined in claim 17, wherein the concentration of azone is about 5%.

31. The topical formulation defined in claim 17, wherein the concentration of azone is about 2%.

32. The topical formulation defined in claim 17, wherein the concentration of azone is about 1%.

33. The topical formulation defined in claim 17, further comprising at least one organic sulfoxide.

34. The topical formulation defined in claim 33, wherein the at least one organic sulfoxide is selected from the group consisting of dimethyl sulfoxide, 1-methylpropyl methyl sulfoxide, 1,1-dimethylpropyl methyl sulfoxide, 1,1-dimethylethyl methyl sulfoxide, 1-methylbutyl methyl sulfoxide, 1,1-dimethylbutyl methyl sulfoxide, 1-ethylbutyl methyl sulfoxide, 1-propylpentyl methyl sulfoxide, trimethylene sulfoxide, 1-propyltrimethylene sulfoxide, 1-butyltrimethylene sulfoxide, thiophene oxide, methyl ethyl sulfoxide, methyl ethylene sulfoxide, 2-hydroxyundecyl methyl sulfoxide, N-decylmethyl sulfoxide and mixtures thereof

35. The topical formulation defined in claim 33, wherein the at least one organic sulfoxide is dimethyl sulfoxide.

36. A topical nail formulation comprising a penetration enhancer selected from the group consisting of a fatty acid, an azone-related compound and mixtures thereof.

37. The topical formulation defined in claim 36, further comprising at least one active agent.

38. The topical formulation defined in claim 36, wherein the fatty acid is selected from the group consisting of is selected from the group consisting of alkanoic acids, capric acid, diacid, ethyloctadecanoic acid, hexanoic acid, lactic acid, lauric acid, linoelaidic acid, linoleic aid, linolenic acid, neodecanoic acid, oleic acid, palmitic acid, pelargonic acid, propionic acid, vaccenic acid and mixtures thereof.

39. The topical formulation defined in claim 36, wherein the fatty acid is oleic acid.

40. The topical formulation defined in claim 36, wherein the azone-related compound is selected from the group consisting of N-acyl-hexahydro-2-oxo-1H-azepines, N-alkyl-dihydro-1,4-oxazepine-5,7-diones, N-alkymorpholine-2,3-diones, N-alkylmorpholine-3,5-diones, azacycloalkane derivatives (-ketone, -thione), azacycloalkenone derivatives, 1-[2-(decylthio)ethyl]azacyclopentan-2-one, N-(2,2-dihydroxyethyl)dodecylamine, 1-dodecanoylhexahydro-1-H-azepine, 1-dodecyl azacycloheptan-2-one, N-dodecyl diethanolamine, N-dodecyl-hexahydro-2-thio-1H-azepine, N-dodecyl-N-(2-methoxyethyl)acetamide, N-dodecyl-N-(2-methoxyethyl)isobutyramide, N-dodecyl-piperidine-2-thione, N-dodecyl-2-piperidinone, N-dodecyl pyrrolidine-3,5-dione, N-dodecyl pyrrolidine-2-thione, N-dodecyl-2-pyrrolidone, 1-farnesylazacycloheptan-2-one, 1-farnesylazacyclopentan-2-one, 1-geranylazacycloheptan-2-one, 1-geranylazacyclopentan-2-one, hexahydro-2-oxo-azepine-1-acetic acid esters, N-(2-hydroxyethyl)-2-pyrrolidone, 1-laurylazacycloheptane, 2-(1-nonyl)-1,3-dioxolane, 1-N-octylazacyclopentan-2-one, N-(1-oxododecyl)-hexahydro-1H-azepine, N-(1-oxododecyl)-morpholines, 1-oxohydrocarbyl-substituted azacyclohexanes, N-(1-oxotetradecyl)-hexahydro-2-oxo-1H-azepine, N-(1-thiododecyl)-morpholines and mixtures thereof.

41. The topical formulation defined in claim 36, wherein the azone-related compound is 1-dodecyl azacycloheptan-2-one.

42. The topical formulation defined in any one of claims 1 through 41, further comprising at least one pharmaceutically acceptable carrier.

43. A topical nail formulation comprising:
(i) between about 1% to about 10% w/w azone;
(ii) between about 1% to about 25% w/w propylene glycol;
(iii) between about 1% to about 15% w/w glycerine;
(iv) between about 1% to about 25% w/w ethanol;
(v) between about 1% to about 80% w/w polyethylene glycol 300; and
(vi) at least one active agent.

44. The topical nail formulation defined in claim 43, wherein the at least one active agent is selected from the group consisting of an antifungal agent, an anti-inflammatory agent, an anti-cancer agent, an antipsoriatic agent and pharmaceutically acceptable salts thereof.

45. The topical nail formulation defined in claim 43, wherein the at least one active agent is an antifungal agent or a pharmaceutically acceptable salt thereof.

46. The topical nail formulation defined in claim 43, wherein the at least one active agents is selected from the group consisting of econazole, terbinafine, butenafine, tolnaftate and pharmaceutically acceptable salts thereof

47. The topical nail formulation defined in claim 43, wherein the at least one active agent is tolnaftate.

48. The topical nail formulation defined in claim 43, wherein the azone is present between about 1% to about 5% by weight of the formulation.

49. A topical nail formulation comprising:
(i) between about 1% to about 10% w/w oleic acid;
(ii) between about 1% to about 25% w/w propylene glycol;
(iii) between about 1% to about 15% w/w glycerine;
(iv) between about 1% to about 25% w/w ethanol;
(v) between about 1% to about 80% w/w polyethylene glycol 300; and
(vi) at least one active agent.

50. The topical nail formulation defined in claim 49, wherein the at least one active agent is selected from the group consisting of an antifungal agent, an anti-inflammatory agent, an anti-cancer agent, an antipsoriatic agent and pharmaceutically acceptable salts thereof.

51. The topical nail formulation defined in claim 49, wherein the at least one active agent is an antifungal agent or a pharmaceutically acceptable salt thereof.

52. The topical nail formulation defined in claim 49, wherein the at least one active agent is selected from the group consisting of econazole, terbinafine, butenafine, tolnaftate and pharmaceutically acceptable salts thereof.

53. The topical nail formulation defined in claim 49, wherein the at least one active agent is tolnaftate or a pharmaceutically acceptable salt thereof.

54. The topical nail formulation defined in claim 49, wherein the oleic acid is present between about 1% to about 5% by weight of the formulation.

55. A method of treating or ameliorating a nail condition comprising the steps of administering to a nail in need of such treatment a therapeutically effective amount of the nail formulation of claim 17.

56. The method defined in claim 55, wherein the nail condition is onychomycosis.

57. A method for enhancing the nail flux of an active agent comprising the steps of administering to a nail the topical formulation of claim 36 with a therapeutically effective amount of an active agent.

58. The method defined in claim 57, wherein the active agent is selected from the group consisting of an antifungal agent, an anti-inflammatory agent, an anti-cancer agent an antipsoriatic agent and pharmaceutically acceptable salts thereof.

59. A method of delivering an active agent into or through the nail comprising the steps of administering to a nail a therapeutically effective amount of the nail formulation of claim 1.
